SEQUENCE LISTING (34 sequences)

SEQ ID NO: 1 - protein, 30 aa, synthetic construct
  REGION 1..30: note = repeat region
GERGAPGFRGPAGPNGIPGEKGPAGERGAP

SEQ ID NO: 2 - protein, 9 aa, synthetic construct
  REGION 1..9: note = C
GAPGPCCGG

SEQ ID NO: 3 - protein, 489 aa, synthetic construct
  REGION 1..489: note = AT16
GERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGAPGPCCGG

SEQ ID NO: 4 - DNA, 1467 bp, synthetic construct
  misc_feature 1..1467: note = AT16
  source 1..1467: mol_type = other DNA
ggagaaaggggggcgcctggctttcgtggtccggcgggtccgaatggcattccgggtgaaaagggtcctgccggtgagcgtggtgctccgggtgagcgcggcgctccgggtttccgcggtcccgcgggtccgaacggcatcccgggagaaaaaggcccagctggcgagcgcggtgcaccgggcgaacgtggtgccccgggcttccgtggcccagcgggtccgaacggtattccgggcgagaaaggtccggcaggtgaacgtggtgcgccaggcgagcgtggtgcgcctggtttcagaggcccagcaggcccaaatggcatccccggtgagaagggcccagccggtgagcgcggggcaccgggtgaacgtggcgcgccgggctttcgcggaccggcgggtccgaacggcatcccgggtgagaagggtccggctggcgagcgtggtgcgccgggtgaacgtggtgcaccgggattccgcggcccggcgggaccgaatggtattccgggtgagaagggtccggcgggcgaacgcggagcaccaggcgaacgcggcgctccgggctttcgcggtccggcgggtccgaatggtatcccgggcgagaagggtcctgccggtgagcgtggtgccccgggcgaacgtggcgctccgggttttcgtggtccggcgggtccgaacggcattccgggtgaaaagggcccagcgggtgagcgtggcgcgccaggcgagagaggtgccccgggttttcgtggcccggcgggtccgaacggcatcccgggtgagaaaggcccggcgggcgaacgtggtgcgccaggcgagagaggtgctccgggtttccgtggcccggctggtccgaacggtattccgggtgaaaagggcccggcgggcgagcgtggcgcgccgggtgagcgtggtgccccaggctttcgtggtccagctggtccgaacggtatcccgggtgaaaaaggtccggcgggtgagcgtggcgcgccgggtgaacgtggtgccccaggcttccgcgggccggcaggtcccaacggtatcccgggcgagaaaggtccggctggcgagcgaggtgccccgggcgaacgtggcgcgccgggcttccgcggtccggcaggcccgaacggtatcccgggcgagaaaggtccggcaggtgagcgtggtgcgccgggtgaacgcggcgctccgggttttcgtggcccggcaggcccaaatggcattccgggcgaaaaaggcccagcgggtgagcgtggtgccccgggtgagcgcggtgcgccgggtttccgcggtccggcgggtccgaatggtattccgggcgaaaaaggcccggcgggcgagcgtggcgctccgggcgaacgtggagcgccaggattccgcggcccggcaggaccgaacggcatcccgggagaaaagggcccggcgggtgaacgtggtgcaccgggagcgcctggtccgtgttgcggtggt

SEQ ID NO: 5 - protein, 498 aa, synthetic construct
  REGION 1..498: note = T16
GERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPRSGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPRSGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPRSGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPRSGPPGPCCGGG

SEQ ID NO: 6 - protein, 480 aa, synthetic construct
  REGION 1..480: note = T16c
GERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAP

SEQ ID NO: 7 - protein, 12 aa, synthetic construct
GPAGPNGIPGEK

SEQ ID NO: 8 - protein, 18 aa, synthetic construct
GPAGPNGIPGEKGPAGER

SEQ ID NO: 9 - protein, 18 aa, synthetic construct
GAPGFRGPAGPNGIPGEK

SEQ ID NO: 10 - protein, 24 aa, synthetic construct
GAPGFRGPAGPNGIPGEKGPAGER

SEQ ID NO: 11 - protein, 129 aa, synthetic construct
GERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGAPGPCCGG

SEQ ID NO: 12 - DNA, 387 bp, synthetic construct
  source 1..387: mol_type = other DNA
ggagaaaggggggcgcctggctttcgtggtccggcgggtccgaatggcattccgggtgaaaagggtcctgccggtgagcgtggtgctccgggtgagcgcggcgctccgggtttccgcggtcccgcgggtccgaacggcatcccgggagaaaaaggcccagctggcgagcgcggtgcaccgggcgaacgtggtgccccgggcttccgtggcccagcgggtccgaacggtattccgggcgagaaaggtccggcaggtgaacgtggtgcgccaggcgagcgtggtgcgcctggtttcagaggcccagcaggcccaaatggcatccccggtgagaagggcccagccggtgagcgcggggcaccgggagcgcctggtccgtgttgcggtggt

SEQ ID NO: 13 - protein, 249 aa, synthetic construct
GERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGAPGPCCGG

SEQ ID NO: 14 - DNA, 747 bp, synthetic construct
  source 1..747: mol_type = other DNA
ggagaaaggggggcgcctggctttcgtggtccggcgggtccgaatggcattccgggtgaaaagggtcctgccggtgagcgtggtgctccgggtgagcgcggcgctccgggtttccgcggtcccgcgggtccgaacggcatcccgggagaaaaaggcccagctggcgagcgcggtgcaccgggcgaacgtggtgccccgggcttccgtggcccagcgggtccgaacggtattccgggcgagaaaggtccggcaggtgaacgtggtgcgccaggcgagcgtggtgcgcctggtttcagaggcccagcaggcccaaatggcatccccggtgagaagggcccagccggtgagcgcggggcaccgggtgaacgtggcgcgccgggctttcgcggaccggcgggtccgaacggcatcccgggtgagaagggtccggctggcgagcgtggtgcgccgggtgaacgtggtgcaccgggattccgcggcccggcgggaccgaatggtattccgggtgagaagggtccggcgggcgaacgcggagcaccaggcgaacgcggcgctccgggctttcgcggtccggcgggtccgaatggtatcccgggcgagaagggtcctgccggtgagcgtggtgccccgggcgaacgtggcgctccgggttttcgtggtccggcgggtccgaacggcattccgggtgaaaagggcccagcgggtgagcgtggcgcgccaggagcgcctggtccgtgttgcggtggt

SEQ ID NO: 15 - protein, 369 aa, synthetic construct
GERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGAPGPCCGG

SEQ ID NO: 16 - DNA, 1107 bp, synthetic construct
  source 1..1107: mol_type = other DNA
ggagaaaggggggcgcctggctttcgtggtccggcgggtccgaatggcattccgggtgaaaagggtcctgccggtgagcgtggtgctccgggtgagcgcggcgctccgggtttccgcggtcccgcgggtccgaacggcatcccgggagaaaaaggcccagctggcgagcgcggtgcaccgggcgaacgtggtgccccgggcttccgtggcccagcgggtccgaacggtattccgggcgagaaaggtccggcaggtgaacgtggtgcgccaggcgagcgtggtgcgcctggtttcagaggcccagcaggcccaaatggcatccccggtgagaagggcccagccggtgagcgcggggcaccgggtgaacgtggcgcgccgggctttcgcggaccggcgggtccgaacggcatcccgggtgagaagggtccggctggcgagcgtggtgcgccgggtgaacgtggtgcaccgggattccgcggcccggcgggaccgaatggtattccgggtgagaagggtccggcgggcgaacgcggagcaccaggcgaacgcggcgctccgggctttcgcggtccggcgggtccgaatggtatcccgggcgagaagggtcctgccggtgagcgtggtgccccgggcgaacgtggcgctccgggttttcgtggtccggcgggtccgaacggcattccgggtgaaaagggcccagcgggtgagcgtggcgcgccaggcgagagaggtgccccgggttttcgtggcccggcgggtccgaacggcatcccgggtgagaaaggcccggcgggcgaacgtggtgcgccaggcgagagaggtgctccgggtttccgtggcccggctggtccgaacggtattccgggtgaaaagggcccggcgggcgagcgtggcgcgccgggtgagcgtggtgccccaggctttcgtggtccagctggtccgaacggtatcccgggtgaaaaaggtccggcgggtgagcgtggcgcgccgggtgaacgtggtgccccaggcttccgcgggccggcaggtcccaacggtatcccgggcgagaaaggtccggctggcgagcgaggtgccccgggagcgcctggtccgtgttgcggtggt

SEQ ID NO: 17 - protein, 609 aa, synthetic construct
GERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGERGAPGFRGPAGPNGIPGEKGPAGERGAPGAPGPCCGG

SEQ ID NO: 18 - DNA, 1827 bp, synthetic construct
  source 1..1827: mol_type = other DNA
ggagaaaggggggcgcctggctttcgtggtccggcgggtccgaatggcattccgggtgaaaagggtcctgccggtgagcgtggtgctccgggtgagcgcggcgctccgggtttccgcggtcccgcgggtccgaacggcatcccgggagaaaaaggcccagctggcgagcgcggtgcaccgggcgaacgtggtgccccgggcttccgtggcccagcgggtccgaacggtattccgggcgagaaaggtccggcaggtgaacgtggtgcgccaggcgagcgtggtgcgcctggtttcagaggcccagcaggcccaaatggcatccccggtgagaagggcccagccggtgagcgcggggcaccgggtgaacgtggcgcgccgggctttcgcggaccggcgggtccgaacggcatcccgggtgagaagggtccggctggcgagcgtggtgcgccgggtgaacgtggtgcaccgggattccgcggcccggcgggaccgaatggtattccgggtgagaagggtccggcgggcgaacgcggagcaccaggcgaacgcggcgctccgggctttcgcggtccggcgggtccgaatggtatcccgggcgagaagggtcctgccggtgagcgtggtgccccgggcgaacgtggcgctccgggttttcgtggtccggcgggtccgaacggcattccgggtgaaaagggcccagcgggtgagcgtggcgcgccaggcgagagaggtgccccgggttttcgtggcccggcgggtccgaacggcatcccgggtgagaaaggcccggcgggcgaacgtggtgcgccaggcgagagaggtgctccgggtttccgtggcccggctggtccgaacggtattccgggtgaaaagggcccggcgggcgagcgtggcgcgccgggtgagcgtggtgccccaggctttcgtggtccagctggtccgaacggtatcccgggtgaaaaaggtccggcgggtgagcgtggcgcgccgggtgaacgtggtgccccaggcttccgcgggccggcaggtcccaacggtatcccgggcgagaaaggtccggctggcgagcgaggtgccccgggcgaacgtggcgcgccgggcttccgcggtccggcaggcccgaacggtatcccgggcgagaaaggtccggcaggtgagcgtggtgcgccgggtgaacgcggcgctccgggttttcgtggcccggcaggcccaaatggcattccgggcgaaaaaggcccagcgggtgagcgtggtgccccgggtgagcgcggtgcgccgggtttccgcggtccggcgggtccgaatggtattccgggcgaaaaaggcccggcgggcgagcgtggcgctccgggcgaacgtggagcgccaggattccgcggcccggcaggaccgaacggcatcccgggagaaaagggcccggcgggtgaacgtggtgcaccgggagaaaggggggcgcctggctttcgtggtccggcgggtccgaatggcattccgggtgaaaagggtcctgccggtgagcgtggtgctccgggtgagcgcggcgctccgggtttccgcggtcccgcgggtccgaacggcatcccgggagaaaaaggcccagctggcgagcgcggtgcaccgggcgaacgtggtgccccgggcttccgtggcccagcgggtccgaacggtattccgggcgagaaaggtccggcaggtgaacgtggtgcgccaggcgagcgtggtgcgcctggtttcagaggcccagcaggcccaaatggcatccccggtgagaagggcccagccggtgagcgcggggcaccgggagcgcctggtccgtgttgcggtggt

SEQ ID NO: 19 - protein, 8 aa, synthetic construct
GPAGERGA

SEQ ID NO: 20 - protein, 12 aa, synthetic construct
GFRGPAGPNGIP

SEQ ID NO: 21 - protein, 15 aa, synthetic construct
GERGAPGERGAPGFR

SEQ ID NO: 22 - protein, 21 aa, synthetic construct
EKGPAGERGAPGERGAPGFRG

SEQ ID NO: 23 - protein, 10 aa, synthetic construct
GPNGIPGEKG

SEQ ID NO: 24 - protein, 13 aa, synthetic construct
KGPAGERGAPGER

SEQ ID NO: 25 - protein, 16 aa, synthetic construct
PAGPNGIPGEKGPAGE

SEQ ID NO: 26 - protein, 22 aa, synthetic construct
AGPNGIPGEKGPAGERGAPGER

SEQ ID NO: 27 - protein, 10 aa, synthetic construct
ERGAPGFRGP

SEQ ID NO: 28 - protein, 13 aa, synthetic construct
RGAPGERGAPGFR

SEQ ID NO: 29 - protein, 15 aa, synthetic construct
GPAGPNGIPGEKGPA

SEQ ID NO: 30 - protein, 23 aa, synthetic construct
NGIPGEKGPAGERGAPGERGAPG

SEQ ID NO: 31 - protein, 10 aa, synthetic construct
PAGPNGIPGE

SEQ ID NO: 32 - protein, 14 aa, synthetic construct
GFRGPAGPNGIPGE

SEQ ID NO: 33 - protein, 18 aa, synthetic construct
RGAPGERGAPGFRGPAGP

SEQ ID NO: 34 - protein, 24 aa, synthetic construct
PGERGAPGFRGPAGPNGIPGEKGP

(19) United States
(12) Patent Application Publication
(10) Pub. No.: US 2024/0262868 A1
(43) Pub. Date: Aug. 8, 2024

(54) POLYPEPTIDES AND USES THEREOF

(71) Applicant: SHANXI JINBO BIO-PHARMACEUTICAL CO., LTD., Taiyuan (CN)

(72) Inventors: Xia Yang, Taiyuan (CN); Zhenrui He, Taiyuan (CN)

(21) Appl. No.: 18/688,056

(22) PCT Filed: Feb. 23, 2023
(86) PCT No.: PCT/CN2023/077950

§ 371 Date: Feb. 29, 2024

(30) Foreign Application Priority Data
Jun. 29, 2022 (CN) 202210761804.8

(51) Int. Cl. C07K 14/00; A61K 8/60; C12N 15/63; C12P 19/34

(52) U.S. Cl. CPC C07K 14/001; A61K 8/606; C12N 15/63; C12P 19/34; C07K 2319/20

(57) ABSTRACT

Polypeptides and uses thereof are provided. The polypeptide includes an N-terminal sequence and a C-terminal sequence, wherein the N-terminal sequence includes one or more repeating units including the amino acid sequence shown in SEQ ID NO. 1. The use of a human structural material for tissue filling and augmentation is provided in the present disclosure, which is expected to have a wide range of applications in the fields such as breast augmentation, rhinoplasty and mid-face filling as it does not require transplantation from the human body, yet has the natural advantages of using human tissue for filling.

This application claims priority to Chinese Patent Application No. 202210761804.8, entitled “POLYPEPTIDES AND USES THEREOF”, filed Jun. 29, 2022, which is incorporated herein by reference in its entirety.

### TECHNICAL FIELD

The present disclosure belongs to the technical field of synthetic biology, and specifically relates to collagen polypeptides, a human body structural material which can be used for tissue filling and augmentation.

### BACKGROUND OF THE INVENTION

Due to genetic factors, nutritional status, age factors, etc., some people will have facial structural defects such as temporal depression, nasal base depression, short and retracted chin, depressed nasal bridge. Some women will also have problems such as breast dysplasia or ptosis and atrophy, which seriously affect the aesthetics. With the continuous improvement of people's living standards and the opening up of their mindset, more and more people begin to pay attention to the cosmetic surgery. At present, a filling surgery can be used to improve the appearance and figure of women, in order to obtain a high straight and beautiful nose shape, a flat and smooth face, and full and round breasts. The filling surgery at the present stage can be mainly divided into prosthesis implantation surgery, autologous tissue transplantation surgery, and injection filling surgery.

In the early breast augmentation surgery and rhinoplasty, silica gel prostheses are often used as implants, because silica gel has a good tissue compatibility, no carcinogenicity, no mutagenicity, no teratogenicity and no other problems, and its tear resistance, hardness, elastic retraction force and other aspects are relatively satisfactory. It has the advantages of easy operation, beautiful appearance, immediate effect, etc. However, due to the static electricity on the surface of the silica gel prosthesis, it is easy to absorb some dust or cilia, which can easily lead to wound infection and will also cause some complications after surgery. For example, complications such as obvious scars near breast augmentation incision, a long distance between breasts, difficulty in lifting, prosthesis rupture, leakage, and capsular contracture may occur, and the probability of prosthesis contracture and rupture increases with the prolongation of prosthesis implantation time. Rhinoplasty is prone to problems such as light transmission of the prosthesis, poor tissue compatibility, hard material, poor touch after implantation, easy displacement, etc. Complications such as nasal swelling, nasal tip dermatitis, and excessive subcutaneous tension may be caused, and the nasal dorsum may not be supported.

With the development of plastic surgery techniques, autologous tissue transplantation has played an increasingly important role in cosmetic surgery. Among them, autologous fat transplantation for breast augmentation, rhinoplasty, and mid-face filling is to transplant the fat particles from the richer parts of the body to desired parts. The autologous fat transplantation is usually performed by injection, which has advantages of less pain, less surgical trauma, shorter time-consuming, no obvious scar in the surgical area, and quick postoperative recovery. The filler for autologous fat transplantation is derived from recipients themselves and has the following properties: is biocompatible and has no immune rejection response after transplantation; will not affect the recipients' own breast or nasal functions; has an excellent fusion capability, and is not easy to shift; fewer postoperative complications; and can also slim down and shape up the recipients. However, the traditional fat collection and injection techniques have shortcomings such as high absorption rate and low survival rate, and excessive one-time transplantation can also cause complications such as breast nodules, so multiple fat transplantations are often required to achieve the desired effect. In addition to autologous fat, autologous cartilage can also be used as a raw material for rhinoplasty and mid-face filling. The autologous cartilage has similar advantages to autologous fat, but it will produce autologous distortion and deformation, which will have a greater impact on the subsequent filling effect.

The injection filling is a surgical procedure in which artificial chemicals are filled into breast or facial defects by injection. Among them, artificial fat is the most commonly used injection filling material, and its chemical composition is hydrophilic polyallylamine hydrogel, which is often used in breast augmentation, but it has been found to cause many complications and adverse reactions in subsequent long-term applications. The hydrogel will contain toxic heavy metals during the production process, which are easily absorbed and accumulated by the human body through the skin and mucous membranes, causing poisoning. It also has varying degrees of toxic and side effects on cells and kidneys. After the injection, the hydrogel will cause complications such as lumps or induration in the breast, pain, bilateral breast asymmetry, infection, lactation mastitis, aseptic inflammation, breast ulceration and perforation, displacement of the injected material, and limited movement of upper limb. Some patients have several complications coexisted.

With the development of modern technology, recombinant collagen can be produced by genetic engineering technology, but the collagen currently on the market is mainly obtained through mutation of the sequence of human collagen, which belongs to collagen-like protein and still has certain immunogenicity. Such collagen still needs to be mixed with a cross-linking agent to prepare a gel product in order to achieve its biomechanical properties and complete tissue augmentation and filling. However, the use of the cross-linking agent often leads to unobservable side effects and reactions to foreign materials produced as collagen with low immunogenicity and non-toxicity is used. Therefore, there is an urgent need in the field to find a humanized collagen having no exogenous gene sequences, non-immunogenicity, high biocompatibility, and the ability to cross-link on its own, and which can be used as a structural material for tissue filling and augmentation.

### SUMMARY OF THE INVENTION

The inventors have conducted long-term research on humanized collagen, and discovered a variety of type III collagen polypeptides in Chinese patent applications CN201210482543.2 and CN201811438582.6. The present inventors further conducted research on these discovered collagen polypeptides, and found that these collagen polypeptides cannot form gels by themselves at a low temperature. Surprisingly, the inventors added small peptide segments to the previously discovered collagen polypeptides, enabling the formed collagen polypeptides to form gels at the low temperature. The gels of the present disclosure may be free of a cross-linking agent.

In one aspect, the present disclosure provides a polypeptide comprising an N-terminal sequence and a C-terminal sequence, wherein the N-terminal sequence comprises one or more repeating units comprising the amino acid sequence shown in SEQ ID NO.1, and the C-terminal sequence is the amino acid sequence shown in SEQ ID NO.2. The amino acid sequence of SEQ ID NO.1 is gergapgfrgpagpngipgekgpagergap. The amino acid sequence of SEQ ID NO.2 is gapgpccgg.

In one embodiment, the repeating unit may be an amino acid sequence obtained after mutation (substitution, insertion, deletion or addition) of one or more amino acid residues in the amino acid sequence of SEQ ID NO.1.

In one embodiment, the C-terminal sequence may be an amino acid sequence obtained after mutation (substitution, insertion, deletion or addition) of one or more amino acid residues in the amino acid sequence of SEQ ID NO.2.

In one embodiment, the number of the repeating unit is 1-20.

In one embodiment, the number of the repeating unit is 2, 3, 4, 5, 6, 7, 8, 9, 10, 11, 12, 13, 14, 15, 16, 17, 18, 19 or 20.

In one embodiment, the N-terminal sequence and the C-terminal sequence are directly linked or separated by one or more amino acid residues.

In one embodiment, each repeating unit is directly linked or separated by one or more amino acid residues;

In one embodiment, the polypeptide comprises an amino acid sequence of SEQ ID NO.3 or an amino acid sequence obtained after mutation (substitution, insertion, deletion or addition) of one or more amino acid residues in the amino acid sequence of SEQ ID NO.3.

In one embodiment, in the case of a mutation in the polypeptide sequences, the obtained polypeptides retain the functions of the present disclosure, such as cell adhesion, the ability to form a gel by itself, and the like.

In one aspect, the present disclosure provides a polynucleotide encoding the polypeptide described herein.

In one embodiment, the polynucleotide comprises the nucleotide sequence shown in SEQ ID NO.4.

In one aspect, the present disclosure provides a nucleic acid comprising a polynucleotide herein.

In one embodiment, the nucleic acid further comprises nucleotides encoding a purification tag, e.g. an His tag, a GST tag, an MBP tag, a SUMO tag, or a NusA tag.

In one embodiment, the nucleic acid further comprises nucleotides encoding a leader sequence.

In one aspect, the present disclosure provides a vector comprising the polynucleotide or the nucleic acid herein. In one embodiment, the vector is an expression vector. In one embodiment, the vector comprises an expression control element, such as a promoter, a terminator and/or an enhancer, operably linked to the polynucleotide or the nucleic acid.

In one aspect, the present disclosure provides a host cell comprising the polynucleotide, the nucleic acid or the vector herein.

In one embodiment, the host cell is a bacterial, fungal or animal cell. In one embodiment, the bacterium is E. coli. In one embodiment, the fungus is a yeast, such as Saccharomyces cerevisiae.

In one aspect, the present disclosure provides a method of producing the polypeptide comprising:
(1) culturing the host cell described herein under a suitable culture condition;
(2) harvesting the host cell and/or the culture medium comprising the polypeptide;
(3) purifying the polypeptide.

In one aspect, the present disclosure provides a composition comprising the polypeptide described herein. The composition may be a composition for tissue filling and/or augmentation.

In one aspect, the present disclosure provides a gel comprising or prepared from the polypeptide described herein. In one embodiment, the gel does not comprise a cross-linking agent. In one embodiment, the gel herein is a human body structural material for use in tissue filling and/or augmentation.

In another aspect, the present disclosure provides a method of preparing a gel comprising the step of storing the polypeptide described herein at a low temperature.

In one embodiment, the low temperature is a temperature ranging from 2 to 8° C. In one embodiment, the low temperature is 2° C., 3° C., 4° C., 5° C., 6° C., 7° C. or 8° C.

In one embodiment, the method of the present disclosure includes the step of storing the polypeptide solution. Preferably, the polypeptide solution is a sodium chloride solution of the polypeptide. The solution may be 50-500 mM NaCl, e.g. 100 mM, 150 mM, 200 mM, 250 mM, 300 mM, 350 mM, 400 mM or 450 mM. In one embodiment, the pH of the sodium chloride solution is above 6, e.g. 6.5, 7, 7.5, 8.0, 8.5, 9 or 9.5.

In yet another aspect, the present disclosure provides the use of the polypeptide, the polynucleotide, the nucleic acid, the host cell, the composition or the gel described herein for increasing cell adhesion or for use as tissue filling and augmentation. For example, the polypeptide, the polynucleotide, the nucleic acid, the host cell, the composition or the gel herein can be used for breast augmentation, rhinoplasty and/or facial filling.

Advantages of the present disclosure include:
1. The present disclosure successfully synthesized a recombinant type III humanized collagen gel at low-temperature for the first time, and the amino acid sequence of the collagen is all derived from the human body itself, without exogenous amino acid sequence;
2. The recombinant type III humanized collagen prepared by the present disclosure can undergo a cross-linking reaction to form a low-temperature gel without the participation of an exogenous cross-linking agent, and has no biological toxicity;
3. The recombinant type III humanized collagen gel prepared at a low-temperature by the present disclosure belongs to humanized collagen, which has good tissue filling and augmentation effects, and has no immune response and strong tissue compatibility when applied to the human body, and can be injected directly into the human body as a structural material;
4. The method of biosynthesizing the recombinant type III humanized collagen low-temperature gel in the present disclosure can be industrially and stably produced on a large scale;
5. The present disclosure provides the use of a human body structural material for tissue filling and augmentation, which is expected to be widely used in fields such as breast augmentation, rhinoplasty and mid-face filling, because it does not require transplantation from the human body but has the natural advantage of human tissue filling.

### DESCRIPTION OF THE DRAWINGS

FIG. 1: Electrophoresis detection results of the purified recombinant type III humanized collagen AT16.

FIG. 2: Results of cell adhesion activity detection of the purified recombinant type III humanized collagen AT16.

FIG. 3: Plasmid map.

FIG. 4: The image during dynamic viscometry of the human collagens T16, TE16c and AT16.

FIG. 5: Electrophoresis detection results of the purified recombinant type III humanized collagens AT4, AT8, AT12, AT16, and AT20.

FIG. 6: The bioadhesive activity of test samples AT4, AT8, AT12, AT16, and AT20.

FIG. 7: The image of gels formed by the recombinant type III humanized collagens AT4, AT8, AT12, AT16, and AT20.

### DETAILED DESCRIPTION

In order to make the purpose, technical solutions and advantages of the present disclosure clearer, the technical solutions in the embodiments of the present disclosure will be clearly and completely described below in conjunction with the embodiments of the present disclosure. It will be obvious that the described embodiments are a part of the embodiments of the present disclosure, but not all of the embodiments. Based on the embodiments of the present disclosure, all other embodiments obtained by those of ordinary skill in the art without making creative work fall within the scope of protection of the present disclosure.

The inventors have conducted long-term research on humanized collagen, and discovered a variety of type III collagen polypeptides in Chinese patent applications CN201210482543.2 and CN201811438582.6. The present inventors further conducted research on these discovered collagen polypeptides, and found that these collagen polypeptides cannot form gels by themselves at a low temperature. Surprisingly, the inventors added small peptide segments to the previously discovered collagen polypeptides, enabling the formed collagen polypeptides to form gels at the low temperature. The gels of the present disclosure may be free of a cross-linking agent. The amino acid sequence of the functional region of the recombinant type III humanized collagen AT16 screened out is: gergapgfrgpagpngipgekgpagergap gergapgfrgpagpngipgekgpagergap gergapgfrgpagpngipgekgpagergap gergapgfrgpagpngipgekgpagergap gergapgfrgpagpngipgekgpagergap gergapgfrgpagpngipgekgpagergap gergapgfrgpagpngipgekgpagergap gergapgfrgpagpngipgekgpagergap gergapgfrgpagpngipgekgpagergap gergapgfrgpagpngipgekgpagergap gergapgfrgpagpngipgekgpagergap gergapgfrgpagpngipgekgpagergap gergapgfrgpagpngipgekgpagergap gergapgfrgpagpngipgekgpagergap gergapgfrgpagpngipgekgpagergap gergapgfrgpagpngipgekgpagergap gapanccgg (SEQ ID NO.3). The underlined amino acid sequence part is a region for ligation to the amino acid sequences of the patent CN201811438582.6 and providing a new functional region.

As used herein, a “polypeptide” refers to a plurality of amino acid residues linked by a peptide bond. As used herein, with respect to a polypeptide or a specific amino acid sequence, the “C-terminal” and “N-terminal” refer to the position relative to the polypeptide or the particular amino acid sequence, specifically at the carboxy-terminal or amino-terminal direction of the polypeptide or the particular amino acid sequence.

Herein, from the N-terminus to the C-terminus, a polypeptide may comprise an N-terminal sequence and a C-terminal sequence. The N-terminal sequence may comprise one or more repeating units comprising the amino acid sequence shown in SEQ ID NO. 1 or the amino acid sequence obtained after mutation (substitution, addition, insertion or deletion) of one or more amino acid residues in the amino acid sequence shown in SEQ ID NO. 1. The number of the repeating unit may be 1-20. For example, the number of the repeating unit is 2, 3, 4, 5, 6, 7, 8, 9, 10, 11, 12, 13, 14, 15, 16, 17, 18, 19 or 20. In particular, the mutation may be a substitution, such as a conservative amino acid substitution. The amino acid sequence of SEQ ID NO.1 is gergapgfrgpagpngipgekgpagergap. In the N-terminal sequence, each repeating unit may be directly linked or separated by one or more amino acid residues.

The C-terminal sequence may be the amino acid sequence shown in SEQ ID NO.2 or the amino acid sequence obtained after mutation (substitution, addition, insertion or deletion) of one or more amino acid residues in the amino acid sequence shown in SEQ ID NO.2. In particular, the mutation may be a substitution, such as a conservative amino acid substitution. The amino acid sequence of SEQ ID NO.2 is gapgpccgg. The N-terminal sequence and the C-terminal sequence may be directly linked or may be separated by one or more amino acid residues, for example 2-10 amino acid residues. For example, the N-terminal and the C-terminal sequence may be separated by 3, 4, 5, 6, 7, 8 or 9 amino acid residues.

In the case of a mutation in the polypeptide sequence or the presence of a spacer sequence, the obtained polypeptide retains the functions of the present disclosure, such as cell adhesion, the ability to form a gel by itself, and the like.

The polypeptide of the present disclosure may be synthetic or may be expressed recombinantly. In the case of recombinant expression, the polypeptide of the present disclosure may be encoded by a polynucleotide. The polynucleotide can be codon-optimized for the host cell in which it is expressed. The polynucleotide encoding the polypeptide may be operably linked to an expression control element, such as a promoter, a terminator and/or an enhancer, to constitute a nucleic acid, or an expression cassette. The nucleic acid may also comprise nucleotides encoding a purification tag, e.g. an His tag, a GST tag, an MBP tag, a SUMO tag, or a NusA tag, or nucleotides encoding a leader sequence to facilitate purification or secretion of the polypeptide.

As used herein, the term “vector” is a nucleic acid delivery vehicle into which a polynucleotide can be inserted. When the vector enables the expression of the protein encoded by the inserted polynucleotide, the vector is called an expression vector. The vector can be introduced into a host cell by transformation, transduction or transfection, so that the genetic material elements it carries can be expressed in the host cell. The vector is well known to those skilled in the art, including but not limited to: a plasmid; a phagemid; a cosmid; an artificial chromosome, such as yeast artificial chromosome (YAC), a bacterial artificial chromosome (BAC) or a P1-derived artificial chromosome (PAC); a phage such as a λ phage or a M13 phage, and an animal viruses and so on. The vector may contain a variety of elements that control expression, including, but not limited to, promoter sequences, transcription initiation sequences, enhancer sequences, selection elements, and reporter genes. In addition, the vector may also contain a replication initiation site. The vector may comprise the nucleic acid of the present disclosure to facilitate introduction into a cell for expression. The vector may comprise an expression control element, such as a promoter, a terminator and/or an enhancer, operably linked to the nucleic acid.

As used herein, the term “host cell” is a cell into which a nucleic acid molecule has been introduced by molecular biology techniques. These techniques include transfection with viral vectors, transformation with plasmid vectors, and accelerated introduction of naked DNA by electroporation, lipofection, and particle guns. The host cell may be eukaryotic or prokaryotic. For example, the eukaryotic cell is a yeast cell, an animal cell and/or an insect cell. The prokaryotic cell may be an E. coli cell.

The present disclosure also provides a method of producing the polypeptide, which comprises: (1) culturing the host cell herein under a suitable culture condition; (2) harvesting the host cell and/or the culture medium comprising the polypeptide; and (3) purifying the polypeptide. The method of the present disclosure may include the step of digesting the tag via an enzyme.

The polypeptide of the present disclosure may be prepared into a composition or a kit. The composition or the kit may be a composition or a kit for tissue filling and/or augmentation. The composition or the kit may also comprise an auxiliary substance. The composition of the present disclosure may be a gel comprising the polypeptide described herein. The gel may be produced autonomously from the polypeptide described herein, without a cross-linking agent. The composition of the present disclosure, particularly the gel, is a human body structural material, e.g. useful for tissue filling and/or augmentation. The gel of the present disclosure may be prepared by a simple method. For example, a method of making a gel may comprise the step of storing the polypeptide described herein at a low temperature. The polypeptide of the present disclosure may achieve self-gelation at a low temperature. The low temperature may be a temperature of 2-8° C., such as 2° C., 3° C., 4° C., 5° C., 6° C., 7° C. or 8° C. The methods of the present disclosure may comprise the step of storing a polypeptide solution. Preferably, the polypeptide solution is an aqueous sodium chloride solution of the polypeptide. The sodium chloride solution may be a 50 mM-500 mM solution. For example, the sodium chloride solution may be 100 mM, 150 mM, 200 mM, 250 mM, 300 mM, 350 mM, 400 mM, or 450 mM. In one embodiment, the pH of the sodium chloride solution is above 6, e.g. 6.5, 7, 7.5, 8.0, 8.5, 9 or 9.5. The concentration of the polypeptide in the solution may be greater than 5 mg/mL, or greater than 10 mg/mL, for example, may be 10 mg/mL, 15 mg/mL, 20 mg/mL, 30 mg/mL, 40 mg/mL, 50 mg/mL, 60 mg/mL, 70 mg/mL, 80 mg/mL, 90 mg/mL, 100 mg/mL, 200 mg/mL, 300 mg/mL or greater concentrations.

### EXAMPLES

The following Examples are provided to illustrate the present disclosure. It should be understood by those skilled in the art that the Examples are merely illustrative and not limiting. The present disclosure is limited only by the scope of the appended claims.

### Example 1: Construction and Expression of the Recombinant Type III Humanized Collagen Gel

1. A large-scale functional region screening and assembly were performed to obtain the gene fragments of interest for the recombinant type III humanized collagen. The amino acid sequence of AT16 is: gergapgfrgpagpngipgekgpagergapgergapgfrgpagpngipgekgpagergapgergapgfrgpa gpngipgekgpagergapgergapgfrgpagpngipgekgpagergapgergapgfrgpagpngipgekgp agergapgergapgfrgpagpngipgekgpagergapgergapgfrgpagpngipgekgpagergapgerga pgfrgpagpngipgekgpagergapgergapgfrgpagpngipgekgpagergapgergapgfrgpagpngi pgekgpagergapgergapgfrgpagpngipgekgpagergapgergapgfrgpagpngipgekgpagerg apgergapgfrgpagpngipgekgpagergapgergapgfrgpagpngipgekgpagergapgergapgfrg pagpngipgekgpagergapgergapgfrgpagpngipgekgpagergapgapgpccgg. In the present disclosure, codon optimization was performed for the codon of E. coli, and the optimized base sequence is

(SEQ ID NO: 4)
GGAGAAAGGGGGGCGCCTGGCTTTCGTGGTCCGGGGGGTCCGAATGGCAT
TCCGGGTGAAAAGGGTCCTGCCGGTGAGCGTGGTGCTCCGGGTGAGCGCG
GCGCTCCGGGTTTCCGCGGTCCCGCGGGTCCGAACGGCATCCCGGGAGAA
AAAGGCCCAGCTGGCGAGCGCGGTGCACCGGGCGAACGTGGTGCCCCGGG
CTTCCGTGGCCCAGCGGGTCCGAACGGTATTCCGGGCGAGAAAGGTCCGG
CAGGTGAACGTGGTGCGCCAGGCGAGCGTGGTGCGCCTGGTTTCAGAGGC
CCAGCAGGCCCAAATGGCATCCCCGGTGAGAAGGGCCCAGCCGGTGAGCG
CGGGGCACCGGGTGAACGTGGCGCGCCGGGCTTTCGCGGACCGGCGGGTC
CGAACGGCATCCCGGGTGAGAAGGGTCCGGCTGGCGAGCGTGGTGCGCCG
GGTGAACGTGGTGCACCGGGATTCCGCGGCCCGGCGGGACCGAATGGTAT
TCCGGGTGAGAAGGGTCCGGCGGGCGAACGCGGAGCACCAGGCGAACGCG
GCGCTCCGGGCTTTCGCGGTCCGGCGGGTCCGAATGGTATCCCGGGCGAG
AAGGGTCCTGCCGGTGAGCGTGGTGCCCCGGGCGAACGTGGCGCTCCGGG
TTTTCGTGGTCCGGCGGGTCCGAACGGCATTCCGGGTGAAAAGGGCCCAG
CGGGTGAGCGTGGCGCGCCAGGCGAGAGAGGTGCCCCGGGTTTTCGTGGC
CCGGCGGGTCCGAACGGCATCCCGGGTGAGAAAGGCCCGGCGGGCGAACG
TGGTGCGCCAGGCGAGAGAGGTGCTCCGGGTTTCCGTGGCCCGGCTGGTC
CGAACGGTATTCCGGGTGAAAAGGGCCCGGCGGGCGAGCGTGGCGCGCCG
GGTGAGCGTGGTGCCCCAGGCTTTCGTGGTCCAGCTGGTCCGAACGGTAT
CCCGGGTGAAAAAGGTCCGGCGGGTGAGCGTGGCGCGCCGGGTGAACGTG
GTGCCCCAGGCTTCCGCGGGCCGGCAGGTCCCAACGGTATCCCGGGCGAG
AAAGGTCCGGCTGGCGAGCGAGGTGCCCCGGGCGAACGTGGCGCGCCGGG
CTTCCGCGGTCCGGCAGGCCCGAACGGTATCCCGGGCGAGAAAGGTCCGG
CAGGTGAGCGTGGTGCGCCGGGTGAACGCGGCGCTCCGGGTTTTCGTGGC
CCGGCAGGCCCAAATGGCATTCCGGGCGAAAAAGGCCCAGCGGGTGAGCG
TGGTGCCCCGGGTGAGCGCGGTGCGCCGGGTTTCCGCGGTCCGGCGGGTC
CGAATGGTATTCCGGGCGAAAAAGGCCCGGCGGGCGAGCGTGGCGCTCCG
GGCGAACGTGGAGCGCCAGGATTCCGCGGCCCGGCAGGACCGAACGGCAT
CCCGGGAGAAAAGGGCCCGGCGGGTGAACGTGGTGCACCGGGAGCGCCTG
GTCCGTGTTGCGGTGGT

2. The synthetic gene fragment was inserted into a pET-28a-Trx-His expression vector to obtain a recombinant expression plasmid.

3. The successfully constructed expression plasmid was transformed into E. coli competent cells BL21 (DE3). The specific process is as follows. (1) The E. coli competent cells BL21 (DE3) were taken out from the ultra-low temperature refrigerator and placed on ice, and when half-thawed, 2 μl of the plasmid to be transformed was taken and added into the E. coli competent cells BL21 (DE3), with mixing slightly 2-3 times. (2) The mixture was placed on ice for 30 min, then heat shocked in a water bath at 42° C. for 45-90 s, and taken out and placed in ice bath on ice for 2 min. (3) The mixture was transferred into a biosafety cabinet, and 700 μl of liquid LB medium was added, and then cultured at 37° C., 220 rpm for 60 min. (4) 200 μl of the bacterial solution was taken and evenly spread on the LB plate containing kanamycin sulfate. (5) The plate was cultured in an incubator at 37° C. for 15-17 h until colonies of uniform size grow out.

4. The single colony of optimized genetically engineered E. coli was pick out and placed in LB liquid medium containing kanamycin, cultured at 37° C., 220 rpm for 5 hours, then cooled down to 16° C., and added with IPTG to a final concentration of 0.5 mM for induction. After incubation for 16 hours, the culture was centrifuged at 6000 rpm, 4° C. for 12 min to collect the bacterial cells.

5. The recombinant humanized type III collagen was purified and enzymatically digested, and the specific process is as follows. (1) The bacteria were resuspended in Tris buffer (25 mM Tris, 200 mM NaCl, 20 mM imidazole, pH=8.0), disrupted by homogenization, centrifuged at 17,000 rpm, 4° C. for 20 minutes, and the supernatant was collected. (2) The proteins were bound using the Ni6FF affinity column, and washed with a washing buffer solution containing 20 mM imidazole (20 mM imidazole, 25 mM Tris, 200 mM NaCl, pH 8.0) to wash out impurity proteins, and the column is eluted with a solution containing 350 mM imidazole (350 mM imidazole, 25 mM Tris, 200 mM NaCl, pH 8.0) to obtain the protein of interest. (3) To the eluted protein sample, an appropriate amount of TEV protease having a His tag was added for digestion at 20° C. for 2 hours to obtain recombinant humanized type III collagen AT16. (4) The mixture of the enzymatically digested recombinant humanized type III collagen AT16 was dialyzed and buffer exchanged into sodium chloride solution, using a 10 kDa dialysis bag. (5) The buffer exchanged recombinant humanized type III collagen AT16 was concentrated to a protein concentration of not less than 10 mg/mL, using a 10 kDa ultrafiltration concentration tube.

6. The specific process for the formation of recombinant humanized type III collagen gel is that: the obtained recombinant humanized type III collagen AT16 was stored in a refrigerator, allowing the protein to be cross-linked at a low temperature environment to form a gel.

7. Detection of the purity of the recombinant type III humanized collagen gel.

The purity of the obtained AT16 protein was detected by SDS-PAGE. The specific process is that: 20 μL of the purified protein solution was taken and 5 μl of 5× protein loading buffer (250 mM of TrisHCl (pH 6.8), 10% SDS, 0.5% bromophenol blue, 50% glycerol, 5% β-mercaptoethanol) was added. The mixture was boiled in boiling water at 100° C. for 5 min, then added into SDS PAGE protein gel at 10 μl per well. After electrophoresis at 150 V for 1 h, the gel was stained with a Coomassie Brilliant Blue Stain (0.1% Coomassie Brilliant Blue R-250, 25% ethanol, 10% glacial acetic acid) for protein staining for 3 min, and then destained with a protein destaining solution (10% acetic acid, 5% ethanol).

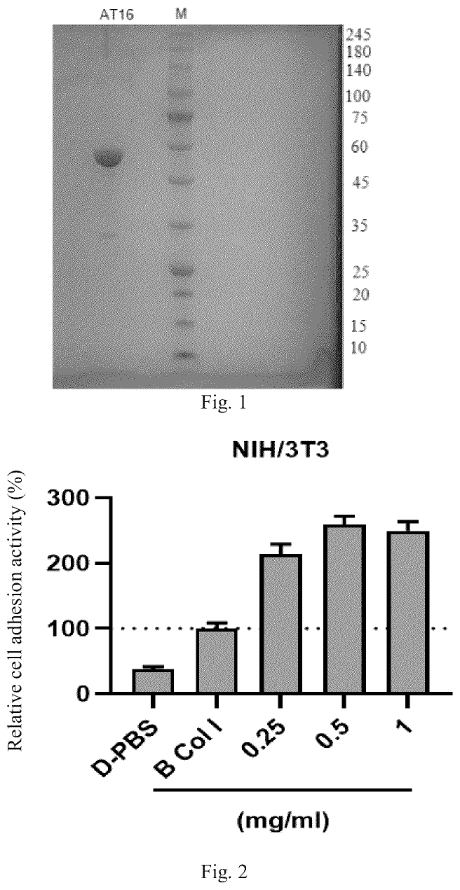

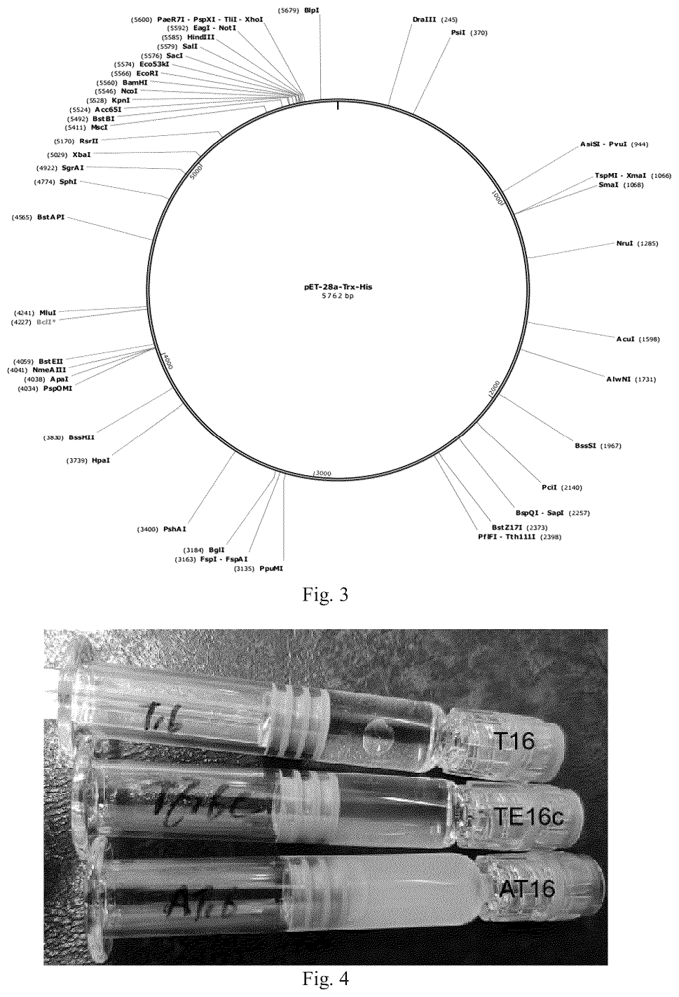

FIG. 1 shows the electrophoresis detection results of the purified recombinant type III humanized collagen AT16. The apparent molecular weight of AT16 is 45 kDa, which corresponds to the theorical weight of the AT16 polypeptide, indicating the correct expression of the polypeptide AT16.

### Example 2: Bioactivity Detection of the Recombinant Type III Humanized Collagen AT16

The collagen activity detection method may be available from the reference, Juming Yao, Satoshi Yanagisawa, Tetsuo Asakura, Design, Expression and Characterization of Collagen-Like Proteins Based on the Cell Adhesive and Crosslinking Sequences Derived from Native Collagens, J Biochem. 136, 643-649(2004). The specific implementation method is as follows:

(1) The concentrations of the protein samples to be tested, including bovine type I collagen standards (Sigma, cat. no.: 380002), and the recombinant type III humanized collagen AT16 provided by the present disclosure, were detected by ultraviolet absorption method.

Specifically, the UV absorption of the samples at 215 nm and 225 nm was measured respectively, and the protein concentrations were calculated using the empirical formula C (μg/mL)=144×(A215−A225). Note that it should be detected under the condition of A215<1.5. The principle of this method is to measure the characteristic absorption of peptide bonds under a far ultraviolet light, which is not affected by the content of chromophores. There are little interfering substances during the process of the method. The method is easy to operate, and is suitable for detecting human collagen and its analogues that are not colored by Coomassie brilliant blue. (reference: Walker J M. The Protein Protocols Handbook, second edition. HumanaPress. 43-45). After detection of the protein concentrations, the concentrations of all proteins to be tested were adjusted to 0.5 mg/mL with PBS.

(2) 100 μL of various protein solutions and blank PBS solution control were added into the 96-well plate respectively, and placed at room temperature for 60 minutes.
(3) 10⁵ well-cultured 3T3 cells were added into each well and incubated at 37° C. for 60 min.
(4) Each well was washed 4 times with PBS.
(5) The absorbance at OD492 nm was detected with LDH detection kit (Roche, cat. no. 04744926001). The cell adhesion rate can be calculated relative to the value of the blank control. The calculation formula is as follows: cell adhesion

$$rate = \frac{test well - blank well}{positive well - blank well} \times 100 \% .$$

The adhesion rate of cells can reflect the activity of collagen. The higher the activity of the protein, the better it can provide the cells with a high-quality external environment in a short time contributing to cell adherence.

The results are shown in FIG. 2 (D-PBS represents the blank PBS group; B ColI represents the bovine type I collagen standards control group; 0.25 represents the AT16 group at 0.25 mg/mL; 0.5 represents the AT16 group at 0.5 mg/mL; 1 represents the AT16 group at 1.0 mg/mL). It can be seen from the comparison that the various concentrations of the recombinant type III humanized collagen AT16 of the present disclosure have better bioadhesive activity, compared to the commercial human collagen.

### Example 3: Mass Spectrometry of the Recombinant Type III Humanized Collagen AT16

Experimental method

| Instrument Name | Matrix Assisted Laser Desorption Ionization- | | |
|---|---|---|---|
| | Time-of-Flight Mass Spectrometry | | |
| | MALDI-TOF/TOF Ultraflextreme ™, | | |
| | Brucker, Germany | | |
| Matrix | CHCA | Laser Energy | 125 |
| Data Search Software | Mascot | Search Species | ALL entries |

| | |
|---|---|
| Search Database | NCBIprot |

The protein samples were reduced with DTT and alkylated with iodoacetamide, and then digested with trypsin overnight. The peptide fragments obtained after enzymatical digestion were desalted with C18ZipTip, followed by mixed with matrix α-cyano-4-hydroxycinnamic acid (CHCA) and plated. Finally, the Matrix-Assisted Laser Desorption Ionization-Time-of-Flight Mass Spectrometer MALDI-TOF/TOF Ultraflextreme™, Brucker, Germany was used for analysis (for the technique of peptide fingerprinting, available from Protein J. 2016; 35:212-7).

Data search was performed through the MS/MS Ion Search page from the local masco website. The protein identification results were obtained based on the primary mass spectrometry of the peptide fragments produced after enzymatical digestion. Detection parameters were: Trypsin digestion, with two missed cut sites. Alkylation of cysteine was set as a fixed modification, and oxidation of methionine as a variable modification. The database used for identification is NCBprot.

TABLE 1
Molecular Weights Detected by Mass
Spectrometry and Corresponding Peptides

| | Mr | |
|---|---|---|
| Observed | (Expected | |
| Value | Value) | Peptide |
| 1093.5637 | 1092.5564 | GPAGPNGIPGEK |
| 1660.8636 | 1659.8563 | GPAGPNGIPGEKGPAGER |
| 1678.8907 | 1677.8834 | GAPGFRGPAGPNGIPGEK |
| 2246.1276 | 2245.1203 | GAPGFRGPAGPNGIPGEK |
| | | GPAGER |

The coverage rate of detected peptide fragments was 84.66%.

### Example 4: Dynamic Viscometry of the Recombinant Type III Humanized Collagen T16, TE16c, and AT16

### Experimental Method

The recombinant type III human collagen T16 (the amino acid sequence is GERGAPGFRGPAGPNGIPGEKGPAGERGAPGERGAPGFRGPAGPNGIPGE KGPAGERGAPGERGAPGFRGPAGPNGIPGEKGPAGERGAPGERGAPGFRG PAGPNGIPGEKGPAGERGAPRSGERGAPGFRGPAGPNGIPGEKGPAGERG APGERGAPGFRGPAGPNGIPGEKGPAGERGAPGERGAPGFRGPAGPNGIP GEKGPAGERGAPGERGAPGFRGPAGPNGIPGEKGPAGERGAPRSGERGAP GFRGPAGPNGIPGEKGPAGERGAPGERGAPGFRGPAGPNGIPGEKGPAGE RGAPGERGAPGFRGPAGPNGIPGEKGPAGERGAPGERGAPGFRGPAGPNG IPGEKGPAGERGAPRSGERGAPGFRGPAGPNGIPGEKGPAGERGAPGERG APGFRGPAGPNGIPGEKGPAGERGAPGERGAPGFRGPAGPNGIPGEKGPA GERGAPGERGAPGFRGPAGPNGIPGEKGPAGERGAPRSGPPGPCCGGG, SEQ ID NO.5) and TE16c (the amino acid sequence is GERGAPGFRGPAGPNGIPGEKGPAGERGAPGERGAPGFRGPAGPNGIPGE KGPAGERGAPGERGAPGFRGPAGPNGIPGEKGPAGERGAPGERGAPGFRG PAGPNGIPGEKGPAGERGAPGERGAPGFRGPAGPNGIPGEKGPAGERGAP GERGAPGFRGPAGPNGIPGEKGPAGERGAPGERGAPGFRGPAGPNGIPGE KGPAGERGAPGERGAPGFRGPAGPNGTPGEKGPAGERGAPGERGAPGFRG PAGPNGIPGEKGPAGERGAPGERGAPGFRGPAGPNGIPGEKGPAGERGAP GERGAPGFRGPAGPNGIPGEKGPAGERGAPGERGAPGFRGPAGPNGIPGE KGPAGERGAPGERGAPGFRGPAGPNGTPGEKGPAGERGAPGERGAPGFRG PAGPNGIPGEKGPAGERGAPGERGAPGFRGPAGPNGIPGEKGPAGERGAP GERGAPGFRGPAGPNGIPGEKGPAGERGAP, SEQ ID NO.6) as well as the recombinant type III humanized collagen AT16 of the present disclosure at the same concentration and in corresponding solution (150 mM NaCl solution, polypeptide concentration 15 mg/mL) were stored in the environment of 2-8° C. respectively and put into the instrument rotational viscometer, in which the dynamic viscosity was tested for comparison.

### Experimental Results

The dynamic viscosities of the recombinant type III humanized collagen T16 and TE16c are unmeasurable, while the measured dynamic viscosity of the recombinant type III humanized collagen AT16 of the present disclosure is 3510 mpa·s. This shows that the recombinant type III humanized collagen AT16 of the present disclosure is able to form a gel better under the same conditions, as shown in FIG. 4. According to FIG. 4, compared to the T16 and TE16c which are colorless and transparent, the AT16 is milky white and is able to form a gel.

### Example 5: Purification and Preparation of AT4, AT8, AT12, AT16, and AT20 Proteins

The AT4, AT8, AT12, AT16, AT20 proteins were prepared and purified as described in Example 1, wherein the number of repeating units of AT4 (gergapgfrgpagpngipgekgpagergap) is 4; the number of repeating units of AT8 is 8; the number of repeating units of AT12 (gergapgfrgpagpngipgekgpagergap) is 12; the number of repeating units of AT16 is 16; and the number of repeating units of AT20 protein is 20.

The specific sequences are as follows:

### The Amino Acid Sequence of AT4

(SEQ ID NO. 11)
Gergapgfrgpagpngipgekgpagergapgergapgfrgpagpngipge
kgpagergapgergapgfrgpagpngipgekgpagergapgergapgfrg
pagpngipgekgpagergapgapgpcegg
The corresponding optimized base sequence
(SEQ ID NO. 12)
GGAGAAAGGGGGGCGCCTGGCTTTCGTGGTCCGGCGGGTCCGAATGGCAT
TCCGGGTGAAAAGGGTCCTGCCGGTGAGCGTGGTGCTCCGGGTGAGCGCG
GCGCTCCGGGTTTCCGCGGTCCCGCGGGTCCGAACGGCATCCCGGGAGAA
AAAGGCCCAGCTGGCGAGCGCGGTGCACCGGGCGAACGTGGTGCCCCGGG
CTTCCGTGGCCCAGCGGGTCCGAACGGTATTCCGGGCGAGAAAGGTCCGG
CAGGTGAACGTGGTGCGCCAGGCGAGCGTGGTGCGCCTGGTTTCAGAGGC
CCAGCAGGCCCAAATGGCATCCCCGGTGAGAAGGGCCCAGCCGGTGAGCG
CGGGGCACCGGGAGCGCCTGGTCCGTGTTGCGGTGGT
The amino acid sequence of AT8
(SEQ ID NO. 13)
Gergapgfrgpagpngipgekgpagergapgergapgfrgpagpngipge
kgpagergapgergapgfrgpagpngipgekgpagergapgergapgfrg
pagpngipgekgpagergapgergapgfrgpagpngipgekgpagergap
gergapgfrgpagpngipgekgpagergapgergapgfrgpagpngipge
kgpagergapgergapgfrgpagpngipgekgpagergapgapgpccgg
The corresponding optimized base sequence
(SEQ ID NO. 14)
GGAGAAAGGGGGGCGCCTGGCTTTCGTGGTCCGGCGGGTCCGAATGGCAT
TCCGGGTGAAAAGGGTCCTGCCGGTGAGCGTGGTGCTCCGGGTGAGCGCG
GCGCTCCGGGTTTCCGCGGTCCCGCGGGTCCGAACGGCATCCCGGGAGAA
AAAGGCCCAGCTGGCGAGCGCGGTGCACCGGGCGAACGTGGTGCCCCGGG
CTTCCGTGGCCCAGCGGGTCCGAACGGTATTCCGGGCGAGAAAGGTCCGG
CAGGTGAACGTGGTGCGCCAGGCGAGCGTGGTGCGCCTGGTTTCAGAGGC
CCAGCAGGCCCAAATGGCATCCCCGGTGAGAAGGGCCCAGCCGGTGAGCG
CGGGGCACCGGGTGAACGTGGCGCGCCGGGCTTTCGCGGACCGGGGGGTC
CGAACGGCATCCCGGGTGAGAAGGGTCCGGCTGGCGAGCGTGGTGCGCCG
GGTGAACGTGGTGCACCGGGATTCCGCGGCCCGGCGGGACCGAATGGTAT
TCCGGGTGAGAAGGGTCCGGCGGGCGAACGCGGAGCACCAGGCGAACGCG
GCGCTCCGGGCTTTCGCGGTCCGGCGGGTCCGAATGGTATCCCGGGCGAG
AAGGGTCCTGCCGGTGAGCGTGGTGCCCCGGGCGAACGTGGCGCTCCGGG
TTTTCGTGGTCCGGCGGGTCCGAACGGCATTCCGGGTGAAAAGGGCCCAG
CGGGTGAGCGTGGCGCGCCAGGAGCGCCTGGTCCGTGTTGCGGTGGT
The amino acid sequence of AT12
(SEQ ID NO. 15)
gergapgfrgpagpngipgekgpagergapgergapgfrgpagpngipge
kgpagergapgergapgfrgpagpngipgekgpagergapgergapgfrg
pagpngipgekgpagergapgergapgfrgpagpngipgekgpagergap
gergapgfrgpagpngipgekgpagergapgergapgfrgpagpngipge
kgpagergapgergapgfrgpagpngipgekgpagergapgergapgfrg
pagpngipgekgpagergapgergapgfrgpagpngipgekgpagergap
gergapgfrgpagpngipgekgpagergapgergapgfrgpagpngipge
kgpagergapgapgpccgg
The corresponding optimized base sequence
(SEQ ID NO. 16)
GGAGAAAGGGGGGCGCCTGGCTTTCGTGGTCCGGCGGGTCCGAATGGCAT
TCCGGGTGAAAAGGGTCCTGCCGGTGAGCGTGGTGCTCCGGGTGAGCGCG
GCGCTCCGGGTTTCCGCGGTCCCGCGGGTCCGAACGGCATCCCGGGAGAA
AAAGGCCCAGCTGGCGAGCGCGGTGCACCGGGCGAACGTGGTGCCCCGGG
CTTCCGTGGCCCAGCGGGTCCGAACGGTATTCCGGGCGAGAAAGGTCCGG
CAGGTGAACGTGGTGCGCCAGGCGAGCGTGGTGCGCCTGGTTTCAGAGGC
CCAGCAGGCCCAAATGGCATCCCCGGTGAGAAGGGCCCAGCCGGTGAGCG
CGGGGCACCGGGTGAACGTGGCGCGCCGGGCTTTCGCGGACCGGCGGGTC
CGAACGGCATCCCGGGTGAGAAGGGTCCGGCTGGCGAGCGTGGTGCGCCG
GGTGAACGTGGTGCACCGGGATTCCGCGGCCCGGGGGACCGAATGGTATT
CCGGGTGAGAAGGGTCCGGCGGGCGAACGCGGAGCACCAGGCGAACGCGG
CGCTCCGGGCTTTCGCGGTCCGGCGGGTCCGAATGGTATCCCGGGCGAGA
AGGGTCCTGCCGGTGAGCGTGGTGCCCCGGGCGAACGTGGCGCTCCGGGT
TTTCGTGGTCCGGCGGGTCCGAACGGCATTCCGGGTGAAAAGGGCCCAGC
GGGTGAGCGTGGCGCGCCAGGCGAGAGAGGTGCCCCGGGTTTTCGTGGCC
CGGCGGGTCCGAACGGCATCCCGGGTGAGAAAGGCCCGGCGGGCGAACGT
GGTGCGCCAGGCGAGAGAGGTGCTCCGGGTTTCCGTGGCCCGGCTGGTCC
GAACGGTATTCCGGGTGAAAAGGGCCCGGCGGGCGAGCGTGGCGCGCCGG
GTGAGCGTGGTGCCCCAGGCTTTCGTGGTCCAGCTGGTCCGAACGGTATC
CCGGGTGAAAAAGGTCCGGCGGGTGAGCGTGGCGCGCCGGGTGAACGTGG
TGCCCCAGGCTTCCGCGGGCCGGCAGGTCCCAACGGTATCCCGGGCGAGA
AAGGTCCGGCTGGCGAGCGAGGTGCCCCGGGAGCGCCTGGTCCGTGTTGC
GGTGGT
The amino acid sequence of AT16
gergapgfrgpagpngipgekgpagergapgergapgfrgpagpngipge
kgpagergapgergapgfrgpagpngipgekgpagergapgergapgfrg
pagpngipgekgpagergapgergapgfrgpagpngipgekgpagergap
gergapgfrgpagpngipgekgpagergapgergapgfrgpagpngipge
kgpagergapgergapgfrgpagpngipgekgpagergapgergapgfrg
pagpngipgekgpagergapgergapgfrgpagpngipgekgpagergap
gergapgfrgpagpngipgekgpagergapgergapgfrgpagpngipge
kgpagergapgergapgfrgpagpngipgekgpagergapgergapgfrg
pagpngipgekgpagergapgergapgfrgpagpngipgekgpagergap
gergapgfrgpagpngipgekgpagergapgapgpccgg
The corresponding optimized base sequence
GGAGAAAGGGGGGCGCCTGGCTTTCGTGGTCCGGCGGGTCCGAATGGCAT
TCCGGGTGAAAAGGGTCCTGCCGGTGAGCGTGGTGCTCCGGGTGAGCGCG
GCGCTCCGGGTTTCCGCGGTCCCGCGGGTCCGAACGGCATCCCGGGAGAA
AAAGGCCCAGCTGGCGAGCGCGGTGCACCGGGCGAACGTGGTGCCCCGGG
CTTCCGTGGCCCAGCGGGTCCGAACGGTATTCCGGGCGAGAAAGGTCCGG
CAGGTGAACGTGGTGCGCCAGGCGAGCGTGGTGCGCCTGGTTTCAGAGGC
CCAGCAGGCCCAAATGGCATCCCCGGTGAGAAGGGCCCAGCCGGTGAGCG
CGGGGCACCGGGTGAACGTGGCGCGCCGGGCTTTCGCGGACCGGCGGGTC
CGAACGGCATCCCGGGTGAGAAGGGTCCGGCTGGCGAGCGTGGTGCGCCG
GGTGAACGTGGTGCACCGGGATTCCGCGGCCCGGCGGGACCGAATGGTAT
TCCGGGTGAGAAGGGTCCGGCGGGCGAACGCGGAGCACCAGGCGAACGCG
GCGCTCCGGGCTTTCGCGGTCCGGGGGTCCGAATGGTATCCCGGGCGAGA
AGGGTCCTGCCGGTGAGCGTGGTGCCCCGGGCGAACGTGGCGCTCCGGGT
TTTCGTGGTCCGGCGGGTCCGAACGGCATTCCGGGTGAAAAGGGCCCAGC
GGGTGAGCGTGGCGCGCCAGGCGAGAGAGGTGCCCCGGGTTTTCGTGGCC
CGGCGGGTCCGAACGGCATCCCGGGTGAGAAAGGCCCGGCGGGCGAACGT
GGTGCGCCAGGCGAGAGAGGTGCTCCGGGTTTCCGTGGCCCGGCTGGTCC
GAACGGTATTCCGGGTGAAAAGGGCCCGGCGGGCGAGCGTGGCGCGCCGG
GTGAGCGTGGTGCCCCAGGCTTTCGTGGTCCAGCTGGTCCGAACGGTATC
CCGGGTGAAAAAGGTCCGGCGGGTGAGCGTGGCGCGCCGGGTGAACGTGG
TGCCCCAGGCTTCCGCGGGCCGGCAGGTCCCAACGGTATCCCGGGCGAGA
AAGGTCCGGCTGGCGAGCGAGGTGCCCCGGGCGAACGTGGCGCGCCGGGC
TTCCGCGGTCCGGCAGGCCCGAACGGTATCCCGGGCGAGAAAGGTCCGGC
AGGTGAGCGTGGTGCGCCGGGTGAACGCGGCGCTCCGGGTTTTCGTGGCC
CGGCAGGCCCAAATGGCATTCCGGGCGAAAAAGGCCCAGCGGGTGAGCGT
GGTGCCCCGGGTGAGCGCGGTGCGCCGGGTTTCCGCGGTCCGGCGGGTCC
GAATGGTATTCCGGGCGAAAAAGGCCCGGCGGGCGAGCGTGGCGCTCCGG
GCGAACGTGGAGCGCCAGGATTCCGCGGCCCGGCAGGACCGAACGGCATC
CCGGGAGAAAAGGGCCCGGCGGGTGAACGTGGTGCACCGGGAGCGCCTGG
TCCGTGTTGCGGTGGT
The amino acid sequence of AT20
gergapgfrgpagpngipgekgpagergapgergapgfrgpagpngipge
kgpagergapgergapgfrgpagpngipgekgpagergapgergapgfrg
pagpngipgekgpagergapgergapgfrgpagpngipgekgpagergap
gergapgfrgpagpngipgekgpagergapgergapgfrgpagpngipge
kgpagergapgergapgfrgpagpngipgekgpagergapgergapgfrg
pagpngipgekgpagergapgergapgfrgpagpngipgekgpagergap
gergapgfrgpagpngipgekgpagergapgergapgfrgpagpngipge
kgpagergapgergapgfrgpagpngipgekgpagergapgergapgfrg
pagpngipgekgpagergapgergapgfrgpagpngipgekgpagergap
gergapgfrgpagpngipgekgpagergapgergapgfrgpagpngipge
kgpagergapgergapgfrgpagpngipgekgpagergapgergapgfrg
pagpngipgekgpagergapgergapgfrgpagpngipgekgpagergap
gapgpccgg
The corresponding optimized base sequence
GGAGAAAGGGGGGCGCCTGGCTTTCGTGGTCCGGCGGGTCCGAATGGCAT
TCCGGGTGAAAAGGGTCCTGCCGGTGAGCGTGGTGCTCCGGGTGAGCGCG
GCGCTCCGGGTTTCCGCGGTCCCGCGGGTCCGAACGGCATCCCGGGAGAA
AAAGGCCCAGCTGGCGAGCGCGGTGCACCGGGCGAACGTGGTGCCCCGGG
CTTCCGTGGCCCAGCGGGTCCGAACGGTATTCCGGGCGAGAAAGGTCCGG
CAGGTGAACGTGGTGCGCCAGGCGAGCGTGGTGCGCCTGGTTTCAGAGGC
CCAGCAGGCCCAAATGGCATCCCCGGTGAGAAGGGCCCAGCCGGTGAGCG
CGGGGCACCGGGTGAACGTGGCGCGCCGGGCTTTCGCGGACCGGCGGGTC
CGAACGGCATCCCGGGTGAGAAGGGTCCGGCTGGCGAGCGTGGTGCGCCG
GGTGAACGTGGTGCACCGGGATTCCGCGGCCCGGCGGGACCGAATGGTAT
TCCGGGTGAGAAGGGTCCGGCGGGCGAACGCGGAGCACCAGGCGAACGCG
GCGCTCCGGGCTTTCGCGGTCCGGCGGGTCCGAATGGTATCCCGGGCGAG
AAGGGTCCTGCCGGTGAGCGTGGTGCCCCGGGCGAACGTGGCGCTCCGGG
TTTTCGTGGTCCGGCGGGTCCGAACGGCATTCCGGGTGAAAAGGGCCCAG
CGGGTGAGCGTGGCGCGCCAGGCGAGAGAGGTGCCCCGGGTTTTCGTGGC
CCGGCGGGTCCGAACGGCATCCCGGGTGAGAAAGGCCCGGGGGCGAACGT
GGTGCGCCAGGCGAGAGAGGTGCTCCGGGTTTCCGTGGCCCGGCTGGTCC
GAACGGTATTCCGGGTGAAAAGGGCCCGGCGGGCGAGCGTGGCGCGCCGG
GTGAGCGTGGTGCCCCAGGCTTTCGTGGTCCAGCTGGTCCGAACGGTATC
CCGGGTGAAAAAGGTCCGGCGGGTGAGCGTGGCGCGCCGGGTGAACGTGG
TGCCCCAGGCTTCCGCGGGCCGGCAGGTCCCAACGGTATCCCGGGCGAGA
AAGGTCCGGCTGGCGAGCGAGGTGCCCCGGGCGAACGTGGCGCGCCGGGC
TTCCGCGGTCCGGCAGGCCCGAACGGTATCCCGGGCGAGAAAGGTCCGGC
AGGTGAGCGTGGTGCGCCGGGTGAACGCGGCGCTCCGGGTTTTCGTGGCC
CGGCAGGCCCAAATGGCATTCCGGGCGAAAAAGGCCCAGCGGGTGAGCGT
GGTGCCCCGGGTGAGCGCGGTGCGCCGGGTTTCCGCGGTCCGGCGGGTCC
GAATGGTATTCCGGGCGAAAAAGGCCCGGCGGGCGAGCGTGGCGCTCCGG
GCGAACGTGGAGCGCCAGGATTCCGCGGCCCGGCAGGACCGAACGGCATC
CCGGGAGAAAAGGGCCCGGCGGGTGAACGTGGTGCACCGGGAGAAAGGGG
GGCGCCTGGCTTTCGTGGTCCGGCGGGTCCGAATGGCATTCCGGGTGAAA
AGGGTCCTGCCGGTGAGCGTGGTGCTCCGGGTGAGCGCGGCGCTCCGGGT
TTCCGCGGTCCCGCGGGTCCGAACGGCATCCCGGGAGAAAAAGGCCCAGC
TGGCGAGCGCGGTGCACCGGGCGAACGTGGTGCCCCGGGCTTCCGTGGCC
CAGCGGGTCCGAACGGTATTCCGGGCGAGAAAGGTCCGGCAGGTGAACGT
GGTGCGCCAGGCGAGCGTGGTGCGCCTGGTTTCAGAGGCCCAGCAGGCCC
AAATGGCATCCCCGGTGAGAAGGGCCCAGCCGGTGAGCGCGGGGCACCGG
GAGCGCCTGGTCCGTGTTGCGGTGGT.

### Example 6: SDS-PAGE Experiment of AT4, AT8, AT12, AT16, and AT20

Experimental process: 20 μL of the purified protein solution was taken and to the protein solution, 5 μl of 5× protein loading buffer (250 mM Tris HCl (pH 6.8), 10% SDS, 0.5% bromophenol blue, 50% glycerol, 5% β-mercaptoethanol) was added. The mixture was boiled in boiling water at 100° C. for 5 min, then added into SDS PAGE protein gel at 10 μl per well. After electrophoresis at 150 V for 1 h, the gel was stained with a Coomassie Brilliant Blue Stain (0.1% Coomassie Brilliant Blue R 250, 25% ethanol, 10% glacial acetic acid) for protein staining for 3 min, and then destained with a protein destaining solution (10% acetic acid, 5% ethanol).

### Experimental Results:

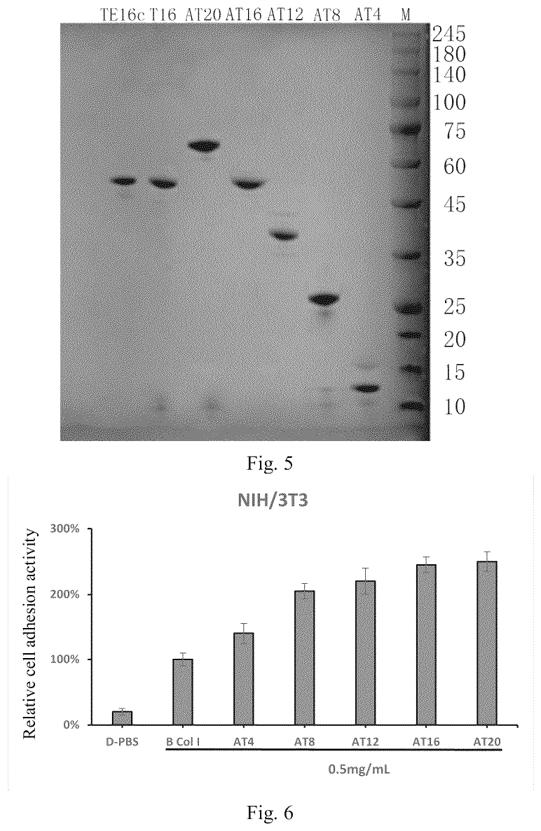

FIG. 5 shows the electrophoresis detection results of the purified recombinant type III humanized collagens AT4, AT8, AT12, AT16 and AT20. The apparent molecular weights of AT4, AT8, AT12, AT16 and AT20 are 13 kDa, 26 kDa, 38 kDa, 45 kDa, and 66 kDa, respectively, and the molecular weights correspond to theorical weights of AT4, AT8, AT12, AT16, and AT20 polypeptides respectively, indicating that AT4, AT8, AT12, AT16, and AT20 polypeptides are correctly expressed.

### Example 7: Bioactivity Detection of AT4, AT8, AT12, AT16, and AT20

### Experimental Process

The collagen activity detection method may be available from the reference, Juming Yao, Satoshi Yanagisawa, Tetsuo Asakura, Design, Expression and Characterization of Collagen-Like Proteins Based on the Cell Adhesive and Crosslinking Sequences Derived from Native Collagens, J Biochem. 136, 643-649(2004). The specific implementation method is as follows:

(1) The concentrations of the protein samples to be tested, including bovine type I collagen standards (Sigma, no.: 380002), and the recombinant type III humanized collagens AT4, AT8, AT12, AT16, and AT20 provided by the present disclosure, were detected by ultraviolet absorption method.

Specifically, the UV absorption of the samples at 215 nm and 225 nm was measured respectively, and the protein concentrations were calculated using the empirical formula C (μg/mL)=144×(A215−A225). Note that it should be detected under the condition of A215<1.5. The principle of this method is to measure the characteristic absorption of peptide bonds under a far ultraviolet light, which is not affected by the content of chromophores. The method has little interfering substances, and is easy to operate, and is suitable for detecting human collagen and its analogues that are not colored by Coomassie brilliant blue. (Reference: Walker J M. The Protein Protocols Handbook, second edition. HumanaPress. 43-45). After detection of the protein concentrations, the concentrations of all proteins to be tested were adjusted to 0.5 mg/mL with PBS.

(2) 100 μL of various protein solutions and blank PBS solution control were added into the 96-well plate respectively, and placed at room temperature for 60 minutes.

(3) 10⁵ well-cultured 3T3 cells were added into each well and incubated at 37° C. for 60 min.

(4) Each well was washed 4 times with PBS.

(5) The absorbance at OD492 nm was detected with LDH detection kit (Roche, cat. no. 04744926001). The cell adhesion rate can be calculated according to the value of the blank control. The calculation formula is as follows:

$$cell adhesion rate = \frac{test well - blank well}{positive well - blank well} \times 100 \% .$$

The adhesion rate of cells can reflect the activity of collagen. The higher the activity of the protein, the better it can provide the cells with a high-quality external environment in a short time contributing to cell adherence.

### Experimental Results

The results are shown in FIG. 6 (D-PBS represents the blank PBS group; B Col I represents the bovine type I collagen standards control group; the concentrations of test samples AT4, AT8, AT12, AT16, and AT20 are respectively 0.5 mg/mL). It can be seen from the comparison that the recombinant type III humanized collagen AT4, AT8, AT12, AT16 and AT20 of the present disclosure all have excellent bioadhesive activities at the concentration of 0.5 mg/mL, compared to commercial human collagen.

### Example 8: Mass Spectrometry of AT4, AT8, AT12, and AT20

### Experimental Method

| Instrument Name | Matrix Assisted Laser Desorption Ionization- | | |
|---|---|---|---|
| | Time-of-Flight Mass Spectrometry | | |
| | MALDI-TOF/TOF Ultraflextreme ™ | | |
| | Brucker, Germany | | |
| Matrix | CHCA | Laser Energy | 125 |
| Data Search Software | Mascot | Search Species | ALL entries |

| | |
|---|---|
| Search Database | NCBIprot |

The protein samples were reduced with DTT and alkylated with iodoacetamide, and then digested with trypsin overnight. The peptide fragments obtained after enzymatical digestion were desalted with C18ZipTip, followed by mixed with matrix α-cyano-4-hydroxycinnamic acid (CHCA) and plated. Finally, the Matrix-Assisted Laser Desorption Ionization-Time-of-Flight Mass Spectrometer MALDI-TOF/TOF Ultraflextreme™, Brucker, Gennany was used for analysis (for the technique of peptide fingerprinting, available from Protein J. 2016; 35:212-7).

Data search is conducted through the MS/MS Ion Search page from the local masco website. The protein identification results were obtained based on the primary mass spectrometry of the peptide fragments produced after enzymatical digestion. Detection parameters were: Trypsin digestion, with two missed cut sites. Alkylation of cysteine was set as a fixed modification, and oxidation of methionine as a variable modification. The database used for identification is NCBprot.

### Experimental Results

| | | |
|---|---|---|
| Observed | Mr (Expected | |
| Value | Value) | Peptide |

AT4 Molecular Weights Detected by Mass
Spectrometry and Corresponding Peptides

| | | |
|---|---|---|
| 839.8683 | 839.8528 | GPAGERGA |
| 1337.4579 | 1337.4432 | GFRGPAGPNGIP |
| 1765.8265 | 1765.8446 | GERGAPGERGAPGFR |
| 2413.5765 | 2413.5232 | EKGPAGERGAPGERGAPGFRG |

AT8 Molecular Weights Detected by Mass
Spectrometry and Corresponding Peptides

| | | |
|---|---|---|
| 1087.1974 | 1087.1434 | GPNGIPGEKG |
| 1497.5287 | 1497.5728 | KGPAGERGAPGER |
| 1602.6752 | 1602.6600 | PAGPNGIPGEKGPAGE |
| 2452.5278 | 2452.5542 | AGPNGIPGEKGPAGERGAPGER |

AT12 Molecular Weights Detected by Mass
Spectrometry and Corresponding Peptides

| | | |
|---|---|---|
| 913.9889 | 913.9748 | ERGAPGFRGP |
| 1442.5276 | 1442.5364 | RGAPGERGAPGFR |
| 1570.6602 | 1570.6612 | GPAGPNGIPGEKGPA |
| 2527.6376 | 2527.6212 | NGIPGEKGPAGERGAPGERGAPG |

AT20 Molecular Weights Detected by Mass
Spectrometry and Corresponding Peptides

| | | |
|---|---|---|
| 1070.1833 | 1070.1130 | PAGPNGIPGE |
| 1559.6287 | 1559.6406 | GFRGPAGPNGIPGE |
| 2013.1492 | 2013.1382 | RGAPGERGAPGFRGPAGP |
| 2686.8492 | 2686.8516 | PGERGAPGFRGPAGPNGIPGEKGP |

### Example 9: Viscosity Testing of AT4, AT8, AT12, AT16, and AT20

### Experiment Process

The recombinant type III humanized collagens AT4, AT8, AT12, AT16, and AT20 of the present disclosure at the same concentration and in corresponding solution (150 mM NaCl solution, polypeptide concentration 15 mg/mL) were stored in the environment of 2-8° C. respectively and put into the instrument rotational viscometer, in which the dynamic viscosity was tested for comparison.

### Experimental Results

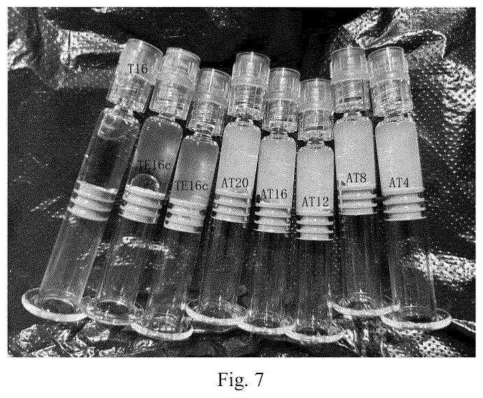

The dynamic viscosities of the recombinant type III humanized collagens T16 and TE16c are unmeasurable, while the measured dynamic viscosities of the recombinant type III humanized collagen AT4, AT8, AT12, AT16 and AT20 of the present disclosure are 3225 mpa·s, 3340 mpa·s, 3475 mpa·s, 3510 mpa·s, and 3650 mpa·s, respectively. This shows that the recombinant type III humanized collagens AT4, AT8, AT12, AT16 and AT20 of the present disclosure are able to form gels better under the same conditions, as shown in FIG. 7. According to FIG. 7, compared to T16 and TE16c which are colorless and transparent, AT4, AT8, AT12, AT16 and AT20 are milky white and are able to form gels.

1. A polypeptide consisting of an N-terminal sequence and a C-terminal sequence, wherein the N-terminal sequence consists of repeating units, wherein the repeating unit consists of the amino acid sequence of SEQ ID NO: 1, wherein the C-terminal sequence consists of the amino acid sequence of SEQ ID NO: 2, wherein the N-terminal sequence and the C-terminal sequence are directly linked, wherein each repeating unit is directly linked, and wherein the number of the repeating units is 4-20.

2. A polynucleotide encoding the polypeptide according to claim 1.

3. A nucleic acid consisting of the polynucleotide according to claim 2 and a nucleotide sequence encoding a purification tag or a nucleotide sequence encoding a leader sequence.

4. A vector comprising the polynucleotide according to claim 2.

5. A host cell comprising the polynucleotide according to claim 2.

6. A method of producing a polypeptide, comprising:
(1) culturing the host cell according to claim 5 under a suitable culture condition;
(2) harvesting the host cell and/or the culture medium comprising the polypeptide; and
(3) purifying the polypeptide.

7. A composition comprising the polypeptide according to claim 1.

8. A gel comprising the polypeptide according to claim 1.

9. A method of preparing a gel, comprising the step of storing the polypeptide according to claim 1 at a temperature ranging from 2-8° C.

10. Use of the polypeptide according to claim 1 for the manufacture of a kit for increasing cell adhesion.

11. The polypeptide of claim 1, which consists of the amino acid sequence of SEQ ID NO: 3.

12. The polynucleotide according to claim 2, consisting of the nucleotide sequence of SEQ ID NO:4.

13. The nucleic acid according to claim 3, wherein the purification tag is a His tag, a GST tag, an MBP tag, a SUMO tag, or a NusA tag.

14. The host cell according to claim 5, wherein the host cell is a bacterium, a fungus, or an animal cell.

15. The host cell according to claim 14, wherein the bacterium is E. coli and the fungus is a yeast.

16. The gel according to claim 8, wherein the gel does not comprise a cross-linking agent.

17. The method according to claim 9, wherein the polypeptide is a polypeptide in a sodium chloride solution.

18. Use of the polypeptide according to claim 1 for use as a kit for tissue filling and/or augmentation.

19. The use according to claim 18, wherein the kit is used for breast augmentation, rhinoplasty and/or facial filling.
